(19) United States
(12) Patent Application Publication
(10) Pub. No.: US 2003/0092125 A1
(43) Pub. Date: May 15, 2003

(54) Epitope-driven human antibody production and gene expression profiling

(75) Inventors: Claude Geoffrey Davis, Burlingame, CA (US); Aya Jakobovits, Menlo Park, CA (US)

Correspondence Address: FISH & NEAVE, 1251 AVENUE OF THE AMERICAS, 50TH FLOOR, NEW YORK, NY, 10020-1105, US

(73) Assignee: Abgenix, Inc., Fremont, CA

(21) Appl. No.: 10/281,387

(22) Filed: Oct. 23, 2002

Related U.S. Application Data
(63) Continuation of application No. 09/060,743, filed on Apr. 15, 1998, now abandoned.

(51) Int. Cl. G01N 33/53; C07H 21/04; C12P 21/02; C12N 5/06; C07K 16/30; C08B 37/00

(52) U.S. Cl. 435/69.1; 435/320.1; 530/388.8; 435/7.1; 435/344; 536/23.53; 536/53

(57) ABSTRACT

The present invention provides a method of biasing the immune response of a mammal toward a desired epitope of a chosen antigen, particularly a functionally-relevant epitope. In preferred embodiments, the epitope-biasing method leads to fully-human antibodies of defined specificity with affinities of 10 nM to 50 pM. The invention further provides antibody libraries biased to tissues and to cell types, for use in generating epitope expression profiles useful for characterizing unknown genes. When all aspects of the present invention are combined, they result in an integrated system for defining critical epitopes on newly discovered gene products and rapidly devloping therapeutic grade antibodies to those critical epitopes.

### FIELD OF THE INVENTION

The present invention is in the field of antibody production and use. In particular, the invention relates to methods and procedures for generating human antibodies of nanomolar and subnanomolar affinity to functionally significant epitopes, which methods include the use of phage display technology. The invention also relates to using a plurality of antibodies and antibody fragments, including human antibodies and fragments thereof, as tissue- and cell type-biased libraries to define epitope expression profiles of newly discovered genes.

### BACKGROUND OF THE INVENTION

In the quarter century since the introduction of hybridoma technology, Kohler et al., Nature 256:495-497 (1975), the immune repertoire of the laboratory mouse has been extensively sampled to provide a wealth of high affinity antibody reagents for in vitro use. But though many of these murine monoclonal antibodies have been raised against antigens of known or presumptive clinical significance, few have yet found use in in vivo diagnostic or therapeutic applications.

An impediment to the in vivo use of murine monoclonal antibodies, early recognized, is that murine antibodies are themselves immunogenic in humans, provoking a human anti-mouse response that limits such fully-murine antibodies to acute therapies. Jaffers et al., Transplant. Proc. 15:643 (1983). A related problem is that murine antibodies do not efficiently recruit cellular elements of the human immune system necessary to effect various desired therapeutic clinical responses.

One approach to solving these problems has been to modify murine monoclonal antibodies of desired antigen specificity through recombinant means, with the goal of reshaping each such antibody to resemble more closely its human counterpart while retaining the original murine binding specificity. Early efforts engrafted a human constant region directly onto the murine antigen-recognizing variable region, to create chimeric antibodies. Cabilly et al., U.S. Pat. No. 4,816,567; Morrison et al., Proc. Natl. Acad. Sci. USA 81:6851-6855 (1984); Boulianne et al., Nature 312:643-646 (1984). More recent attempts have with greater precision introduced the murine variable region complementarity determining regions (CDRs) into human variable region frameworks to create CDR-grafted humanized, or reshaped, antibodies. U.S. Pat. No. 5,530,101; Riechmann et al., Nature 332:323-327 (1988).

Monoclonal antibodies approved to date for in vivo therapeutic use in the United States reflect each of the variants of this approach. OKT3, a fully murine antibody, is approved only for therapeutic intervention in acute transplant rejection. Rituxan (rituximab) and Reopro (abciximab) are chimeric antibodies, the former with specificity for CD20, approved for treatment of low-grade non-Hodgkin's lymphoma recurrences, the latter an inhibitor of platelet aggregation, approved for use in reducing acute ischemic cardiac complications during angioplasty. Zenapax (daclizumab), a CDR-grafted humanized antibody with specificity for the IL-2 receptor, is approved for treatment of acute renal graft rejection. Other murine, chimeric, and humanized antibodies are presently in clinical trials.

Another approach to generating antibodies with in vivo utility has been to create fully-human antibodies, using either phage display or human antibody-transgenic animals.

Human immunoglobulin heavy chain and light chain variable regions may be cloned, combinatorially reasserted, expressed and displayed as antigen-binding human Fab or scfv (“single chain variable region”) fragments on the surface of filamentous phage (“human phAbs”). Rader et al., Current Opinion in Biotechnology 8:503-508 (1997); Aujame et al., Human Antibodies 8:155-168 (1997); Hoogenboom, Trends in Biotechnol. 15:62-70 (1997); de Kruif et al., 17:453-455 (1996); Barbas et al., Trends in Biotechnol. 14:230-234 (1996); Winter et al., Ann. Rev. Immunol. 433-455 (1994). The phage-displayed human antigen-binding fragments may then be screened for their ability to bind a chosen antigen.

It has already been demonstrated, using such human phage display libraries, that it is possible to identify human phAbs that recognize novel epitopes of antigens of known clinical relevance. Thus, Nissim et al., using a library of phage displaying semisynthetic human scFv, identified a scFV with specificity for a novel epitope of the tumor suppressor p53. EMBO J. 13(3):692-698 (1994). It has further been demonstrated that human phAbs with specificity for clinically significant, yet immunologically nondominant, epitopes can be selected from a natural human library. Tsui et al., J. Immunol. 157:772-780 (1996).

Phage display presents problems, however, when high affinity human antibodies are desired. To generate high (nanomolar or subnanomolar) affinity phAbs, three approaches may be pursued.

First, the library may be constructed from an individual who has previously been immunized against the chosen antigen—either by fortuitous prior exposure, Tsui et al.; Ditzel et al., J. Immunol. 154:893 (1995), or through an earlier directed therapeutic intervention, Cai et al., Proc. Natl. Acad. Sci. USA 92:6537-6541 (1995). The requirement for prior immunization of a human donor substantially limits the antigens that may be addressed using this approach.

Second, a synthetic or semisynthetic library may be constructed with sufficient complexity—that is, with a sufficient number of original clones—as to allow such affinity to be obtained by purely random combination. Aujame et al., Human Antibodies 8:155-168 (1997); Griffiths et al., EMBO J. 13:3245 (1994). This approach presents technical difficulties that are only now being addressed.

Finally, lower affinity phAbs selected from a phage display antibody library may be individually modified to increase affinity, through one of a variety of artificial affinity maturation techniques. Yang et al., J. Mol. Biol. 254:392-403 (1995); Schier et al., J. Mol. Biol. 263:551-567 (1996); Thompson et al., J. Mol. Biol. 256:77-88 (1996); Ohlin et al., Mol. Immunol. 33:47-56 (1995). These techniques, like those used to humanize a murine antibody, are tedious and must be repeated individually for each selected antibody.

A separate solution to generating fully human antibodies of high affinity and in vivo utility has been to create strains of transgenic mammals that produce human antibodies in vivo (human antibody-transgenic mammals). In one such variant, termed the Xenomouse™, the endogenous murine Ig heavy and light chain loci have been inactivated by site-directed homologous recombination, and substantially comprehensive portions of the human loci in near-germline configuration introduced on yeast artificial chromosomes. Mendez et al., Nature Genetics 15:146-156 (1997); Jakobovits, Curr. Opin. Biotechnol. 6:561-566 (1995); WO 96/34096; WO 96/33735; WO 94/02602; WO 91/10741. In another variant, the endogenous murine Ig loci have been inactivated and portions of the human Ig loci introduced on small recombinant constructs. U.S. Pat. Nos. 5,661,016; 5,633,425; 5,625,126; 5,569,825; 5,545,806.

Fully human antibodies of high affinity may readily be obtained to a range of antigens using such human antibody-transgenic mice. Immunizing such mice with desired immunogens, using protocols well-established for standard laboratory strains, permits the creation of high affinity, fully-human monoclonal antibodies, using standard hybridoma technology. Such antibodies frequently have affinities in the nanomolar range, and often have affinities in the subnanomolar range.

WO 96/33735 further suggests that the advantage of in vivo affinity maturation in immunized human antibody-transgenic mice may be combined with the combinatorial and screening advantages of phage display by creating phage display antibody libraries from the B cells of such human antibody-transgenic mice after directed immunization.

Although the recombinant reshaping of mouse antibodies and the various approaches to generating fully human antibodies answer the need for agents that are compatible with in vivo administration, none of these techniques fully answers the need to direct such agents to functionally- or clinically-relevant epitopes. Despite intensive efforts, many antigens of known clinical relevance have proven poorly immunogenic, or have failed to elicit murine monoclonal antibodies directed to functionally-relevant epitopes.

It has long been known, for example, that certain epitopes prove immunodominant in the course of a natural immune response; that is, the immune response is directed primarily and reproducibly at particular structures displayed on the immunogen. Green et al., Cell 28(3):477-487; Shinnick et al., Annu. Rev. Microbiol. 37:425-446 (1983). At least one pathogen has been shown to exploit this limitation of the natural immune system: respiratory syncytial virus (RSV) presents an immunologically dominant epitope to the human immune system that leads to vigorous, yet futile, production of non-neutralizing antibodies. Tsui et al., J. Immunol. 157:772-780 (1996). The viral strategy presents clear problems for vaccine development.

The issue of immunodominant epitopes also presents problems in efforts to identify human tumor-associated antigens by immunization of standard mouse strains: the myriad xenogeneic epitopes presented by human tumor cells are preferentially recognized by the murine immune system, and often swamp efforts to identify with specificity tumor-associated changes in cell-surface phenotype. Cai et al., Proc. Natl. Acad. Sci. USA 92:6537-6541 (1995).

One solution to the inherent bias of the immune system has been to drive the immune response toward selected, and occasionally nonimmunodominant, epitopes, through immunization of mice with synthetic peptides conjugated to carriers. In this way, antibodies can be generated to any chosen linear epitope on a protein. Shinnick et al., Annu. Rev. Microbiol. 37:425-446 (1983); Atassi et al., Crit. Rev. Immunol. 5:387-409 (1985). This solution, however, presupposes prior knowledge of the identity and amino acid sequence of the desired epitope, and provides no means for identifying which epitopes are functionally significant.

There is a need in the art, therefore, for means of identifying clinically-relevant epitopes of new or known antigens, and for a method of driving the generation of fully-human antibodies to such specific epitopes.

Recent technical advances in measuring gene expression have made possible the contemporaneous measurement of the expression of many, if not all, genes transcribed in a eukaryotic cell. Lashkari et al., Proc. Natl. Acad. Sci. USA 94:13057-13062 (1997); DeRisi et al., Science 278: 680-686 (1997); Wodicka et al., Nature Biotechnology 15:1359-1367 (1997); Pietu et al., Genome Research 6:492-503 (1996) (hereinafter “Pietu et al.”);

In contrast to the foregoing methods, all of which assay nucleic acid transcript levels, Ashby et al., U.S. Pat. No. 5,549,588 (hereinafter “Ashby et al.”), measure a later stage in expression. Ashby et al. disclose a “genome reporter matrix” in which, in one embodiment, each element of the spatially-addressable matrix consists of a cell (or clone of cells), rather than nucleic acids. The cells at each matrix location contain a recombinant construct that directs expression, from a distinct transcriptional regulatory element, of a common reporter gene. Signals from the reporter indicate expression operably controlled by the respective transcriptional regulatory element, the identity of which is encoded in the spatial location of the element in the matrix.

The foregoing methods report complementary measures of a given gene's expression in a cell: levels of the mRNA transcript on the one hand, and intracellular levels of an encoded translation product on the other. None of these methods, however, reports the availability of immunogenic epitopes on the gene's expression product, and as a result, none of the foregoing methods provides information about the suitability of the respective expression products for diagnostic or therapeutic targeting by antibody reagents. Nor do such existing methods provide an easy route to such diagnostic or therapeutic antibodies.

### SUMMARY OF THE INVENTION

In view of the foregoing, it is an object of this invention to provide a method of biasing the immune response of a mammal toward a desired epitope of a chosen antigen, comprising the steps of (a) selecting, from a phage-displayed antibody library, at least one phage-displayed antibody (phAb) that binds to said antigen; then selecting, in step (b), at least one phage-displayed peptide from a phage-displayed peptide library that binds to the antigen-specific phAb and that mimics a desired epitope of the chosen antigen; and then, in a final step (c), immunizing a mammal with the peptide mimic, thereby biasing the immune response of the mammal to the desired epitope of the chosen antigen.

In one embodiment, this method further comprises at least one iteration of the subsequent steps of (d) constructing a phage-displayed antibody library from immunoglobulin transcripts of the peptide mimic-immunized mammal; followed in order by steps (a) through (c). The iteration further biases the immune response of the mammal to the desired epitope of the chosen antigen.

In a particularly preferred embodiment of the method, the method further comprises the step, after step (a) and before step (b), of further selecting from the phAbs selected in step (a), for further use in step (b), only those phAbs that functionally affect said antigen, biasing the immune response toward a desired functional epitope of a chosen antigen.

The invention further provides, when the phage-displayed antibody library is constructed from a human antibody-transgenic mouse, a method of making a human antibody that is specific for a desired epitope of a chosen antigen, comprising the steps of: (a) biasing the immune response of a human antibody-transgenic mouse toward said epitope, and then (b) isolating an antibody from the transgenic mouse that is specific for said epitope of said antigen.

The invention provides human antibodies that are specific for a desired epitope of a chosen antigen, produced by the above-described process, and in particular, provides human antibodies to L-selectin that function to inhibit the binding of lymphocytes to endothelial venules and human antibodies specific for an epitope of a melanoma-associated antigen.

In another aspect, the invention also provides a spatially-addressable library of antibodies or antigen-binding antibody fragments, wherein said antibodies or antibody fragments derive from a mammal with immune response biased according to the claimed method. In a preferred embodiment, the spatially-addressable library is constructed from antigen-binding fragments of human antibodies.

When all aspects of the present invention are combined, they result in an integrated system for defining critical epitopes on newly discovered gene products and rapidly devloping therapeutic grade antibodies to those critical epitopes.

### BRIEF DESCRIPTION OF THE DRAWINGS

FIG. 1 schematizes a method for biasing the immune response of a mouse to a particular epitope of a chosen antigen.

FIG. 2 demonstrates construction of a scFv antibody library that preferentially includes heavy chain variable regions from gamma transcripts.

FIG. 3 schematizes a method for biasing the immune response of a mouse to a functionally-relevant epitope of a chosen antigen.

### DETAILED DESCRIPTION OF THE INVENTION

In order that the invention herein described may be fully understood, the following detailed description is set forth. In the description, the following terms are employed.

“Antibody-transgenic mammal” denotes a mammal that possesses in its genome—that is, has integrated into the chromosomes of at least some of its somatic cells—a sufficient number of the antibody genes of a heterologous mammalian species to be capable of producing antibody molecules characteristic of the heterologous species. The phrase explicitly includes, but is not limited to: (a) mammals that remain capable of producing endogenous antibody; (b) mammals that are transgenic exclusively for Ig light chains, either Igκ, Igλ, or both; (c) mammals that are transgenic exclusively for at least one Ig heavy chain constant region; (d) mammals that are transgenic for both Ig heavy chains and light chains; (e) mammals that are capable of producing heterologous IgM only, heterologous IgG only, or both IgM and at least one subclass of IgG; (f) mammals heterozygous for the introduced transgenes; (g) mammals homozygous for the introduced transgenes; (h) mammals in which the transgenes are present in germ cells.

The phrase “human antibody transgenic mammal” refers to a subset of “antibody transgenic mammals” in which a nonhuman mammalian species possesses in its genome at least some human antibody genes and is capable of producing antibody molecules characteristic of the human immune system.

The phrase “human antibody transgenic mouse” refers to a subset of “human antibody transgenic mammals” in which a mouse possesses in its genome at least some human antibody genes and is capable of producing antibody molecules characteristic of the human immune system.

The term “Xenomouse™” refers to a subset of human antibody transgenic mice as further described in Mendez et al., Nature Genetics 15:146-156 (1997); Jakobovits, Curr. Opin. Biotechnol. 6:561-566 (1995); WO 96/34096; WO 96/33735; WO 94/02602; WO 91/10741.

The term “bias”, as used with reference to a humoral immune response of a mammal, here denotes an increased representation, as compared to an unimmunized control, in a collection of antibodies or antibody fragments, of antibodies or antibody fragments that bind to a chosen immunogen, antigen, or antigenic epitope. The increased representation may be manifested by any one or more of the following: (a) by the percentage of splenic transcripts that encode antibody chains that bind to a chosen immunogen, antigen, or desired epitope thereof; (b) by the percentage of antibodies detectable in a mammal that bind to a chosen immunogen, antigen, or desired epitope thereof; (c) by the percentage of clones in a phage display antibody library that bind to a chosen immunogen, antigen, or desired epitope thereof; (d) by the percentage of hybridomas resulting from a fusion event that bind to chosen immunogen, antigen, or desired epitope thereof. It will be understood by those skilled in the art of immunology that an increased representation of antibodies that bind to a chosen immunogen, antigen, or epitope thereof will often be accompanied by a concomitantly increased representation of antibodies with higher affinity thereto.

The phrase “epitope-biased immune libraries” refers to a collection of antibodies or antibody fragments with an increased representation, as compared to an unimmunized control, of antibodies or antibody fragments that bind to a desired epitope of a chosen antigen.

As used herein, the phrase “epitope expression profile” denotes a data set, specific for a given protein, each data point of which reports a measure of the binding of the protein to a distinct library of antibodies.

The generation of fully human antibodies, for example, from transgenic animals, is very attractive. Fully human antibodies are expected to minimize the immunogenic and allergic responses intrinsic to mouse or mouse-derived Mabs and thus to increase the efficacy and safety of the administered antibodies. The use of fully human antibodies can be expected to provide a substantial advantage in the treatment of chronic and recurring human diseases, such as inflammation, autoimmunity, and cancer, which often require repeated antibody administrations.

One approach that has been utilized in connection with the generation of human antibodies is the construction of mouse strains that are deficient in mouse antibody production but that possess large fragments of the human Ig loci so that such mice would produce a large repertoire of human antibodies in the absence of mouse antibodies. Large human Ig fragments preserve the large variable gene diversity as well as the proper regulation of antibody production and expression. By exploiting the mouse machinery for antibody diversification and selection and the lack of immunological tolerance to human proteins, the reproduced human antibody repertoire in these mouse strains yields high affinity antibodies against any antigen of interest, including human antigens. Using hybridoma technology, antigen-specific human Mabs with the desired specificity can be readily produced and selected.

This general strategy was demonstrated in connection with the generation of the first XenoMouse strains as published in 1994. See Green et al. Nature Genetics 7:13-21 (1994). The XenoMouse strains were engineered with 245 kb and 190 kb-sized germline configuration fragments of the human heavy chain loci and kappa light chain loci, respectively, which contained core variable and constant region sequences. Id. The human Ig containing yeast artificial chromosomes (YACs) proved to be compatible with the mouse system for both rearrangement and expression of antibodies, and were capable of substituting for the inactivated mouse Ig genes. This was demonstrated by their ability to induce B-cell development and to produce an adult-like human repertoire of fully human antibodies and to generate antigen-specific human Mabs. These results also suggested that introduction of larger portions of the human Ig loci containing greater numbers of V genes, additional regulatory elements, and human Ig constant regions might recapitulate substantially the full repertoire that is characteristic of the human humoral response to infection and immunization.

In Mendez et al. Nature Genetics 15:146-156 (1997), such approach was extended through the introduction of a 1,020 kb heavy chain construct and a 800 kb light chain construct. The heavy chain construct contained approximately 66 V_{H} genes and all of the D and J_{H} genes and the Cμ and Cδ constant regions in germ line configuration and also contained a gamma constant region and mouse heavy chain enhancer. The light chain construct contained approximately 32 Vκ genes (the distal portion of the Vκ locus in germ line configuration) with all of the Jκ genes, the κ constant region, and the kappa deleting element in germ line configuration. Transgenic mice containing such transgenes appear to substantially possess the full human antibody repertoire that is characteristic of the human humoral response to infection and immunization. Such mice are referred to as XenoMouse™ animals.

Such approaches are further discussed and delineated in U.S. patent application Ser. No. 07/466,008, filed Jan. 12, 1990, Ser. No. 07/610,515, filed Nov. 8, 1990, Ser. No. 07/919,297, filed Jul. 24, 1992, Ser. No. 07/922,649, filed Jul. 30, 1992, filed 08/031,801, filed Mar. 15, 1993, Ser. No. 08/112,848, filed Aug. 27, 1993, Ser. No. 08/234,145, filed Apr. 28, 1994, Ser. No. 08/376,279, filed Jan. 20, 1995, Ser. No. 08/430,938, Apr. 27, 1995, Ser. No. 08/464,584, filed Jun. 5, 1995, Ser. No. 08/464,582, filed Jun. 5, 1995, Ser. No. 08/463,191, filed Jun. 5, 1995, Ser. No. 08/462,837, filed Jun. 5, 1995, Ser. No. 08/486,853, filed Jun. 5, 1995, Ser. No. 08/486,857, filed Jun. 5, 1995, Ser. No. 08/486,859, filed Jun. 5, 1995, Ser. No. 08/462,513, filed Jun. 5, 1995, Ser. No. 08/724,752, filed Oct. 2, 1996, and Ser. No. 08/759,620, filed Dec. 3, 1996. See also European Patent No. EP 0 463 151 B1, grant published Jun. 12, 1996. International Patent Application No. WO 94/02602, published Feb. 3, 1994, International Patent Application No. WO 96/34096, published Oct. 31, 1996, and PCT Application No. PCT/US96/05928, filed Apr. 29, 1996. The disclosures of each of the above-cited patents and applications are hereby incorporated by reference in their entirety.

In an alternative approach, others, including GenPharm International, Inc., have utilized a “minilocus” strategy. In the minilocus strategy, an exogenous Ig locus is mimicked through the inclusion of pieces (individual genes) from the Ig locus. Thus, one or more V_{H} genes, one or more D_{H} genes, one or more J_{H} genes, a mu constant region. and a second constant region (preferably a gamma constant region) are formed into a construct for insertion into an animal. This approach is described in U.S. Pat. No. 5,545,807 to Surani et al., U.S. Pat. Nos. 5,545,806, 5,625,825, 5,661,016, 5,633,425, and 5,625,126, each to Lonberg and Kay. U.S. Pat. No. 5,643,763 to Dunn and Choi. U.S. Pat. No. 5,612,205 to Kay et al., U.S. Pat. No. 5,591,669 to Krimpenfort and Berns, and GenPharm International U.S. patent application Ser. Nos. 07/574,748, filed Aug. 29, 1990, Ser. No. 07/575,962, filed Aug. 31, 1990, Ser. No. 07/810,279, filed Dec. 17, 1991, Ser. No. 07/853,408, filed Mar. 18, 1992, Ser. No. 07/904,068, filed Jun. 23, 1992, Ser. No. 07/990,860, filed Dec. 16, 1992, Ser. No. 08/053,131, filed Apr. 26, 1993, Ser. No. 08/096,762, filed Jul. 22, 1993, Ser. No. 08/155,301, filed Nov. 18, 1993, Ser. No. 08/161,739, filed Dec. 3, 1993, Ser. No. 08/165,699, filed Dec. 10, 1993, Ser. No. 08/209,741, filed March 9, 1994, Ser. No. 08/544,404, filed Oct. 10, 1995, the disclosures of which are hereby incorporated by reference. See also International Patent Application Nos. WO 97/13852, published Apr. 17, 1997. WO 94/25585, published Nov. 10, 1994, WO 93/12227, published Jun. 24, 1993, WO 92/22645, published Dec. 23, 1992, WO 92/03918, published Mar. 19, 1992, the disclosures of which arc hereby incorporated by reference in their entirety. See further Taylor et al., 1992, Chen et al., 1993, Tuaillon et al., 1993, Choi et al., 1993, Lonberg et al., (1994), Taylor et al., (1994), and Tuaillon et al., (1995), the disclosures of which are hereby incorporated by reference in their entirety.

The inventors of Surani et al., cited above, and assigned to the Medical Research Counsel (the “MRC”), produced a transgenic mouse possessing an Ig locus through use of the minilocus approach. The inventors on the GenPharm International work, cited above, Lonberg and Kay, following the lead of the present inventors, proposed inactivation of the endogenous mouse Ig locus coupled with substantial duplication of the Surani et al. work.

An advantage of the minilocus approach is the rapidity with which constructs including portions of the Ig locus can be generated and introduced into animals. Commensurately, however, a significant disadvantage of the minilocus approach is that, in theory, insufficient diversity is introduced through the inclusion of small numbers of V, D, and I genes. Indeed, the published work appears to support this concern. B-cell development and antibody production of animals produced through use of the minilocus approach appear stunted. Therefore, the present inventors have consistently urged introduction of large portions of the Ig locus in order to achieve greater diversity and in an effort to reconstitute the immune repertoire of the animals.

As will be appreciated, transgenic non-human mammals that are produced in accordance with the approach utilized to produce XenoMouse animals or the “minilocus” approach are members of the “human antibody transgenic mammal” definition used herein. It will be appreciated that through use of the above-technology, human antibodies can be generated against a variety of antigens, including cells expressing antigens, isolated forms of antigens, epitopes or peptides of such antigens, and expression libraries thereto (see e.g. U.S. Pat. No. 5,703,057) through immunization of a “human antibody transgenic mammal” with the desired antigen or antigens, forming hybridomas, and screening the resulting hybridomas using conventional techniques that arc well known in the art. Such hybridomas that are generated can be utilized in a “panel of antibody moieties” or a “tissue biased library” as described herein in a similar manner as phage libraries can be used. Alternatively, antibodies, or the genetic materials encoding such antibodies, that are secreted by such hybridomas can also be utilized in a “panel of antibody moieties” or “tissue biased library” as described herein. Further, the supernatants of the hybridomas can also be utilized in a “panel of antibody moieties” or “tissue biased library” as described herein.

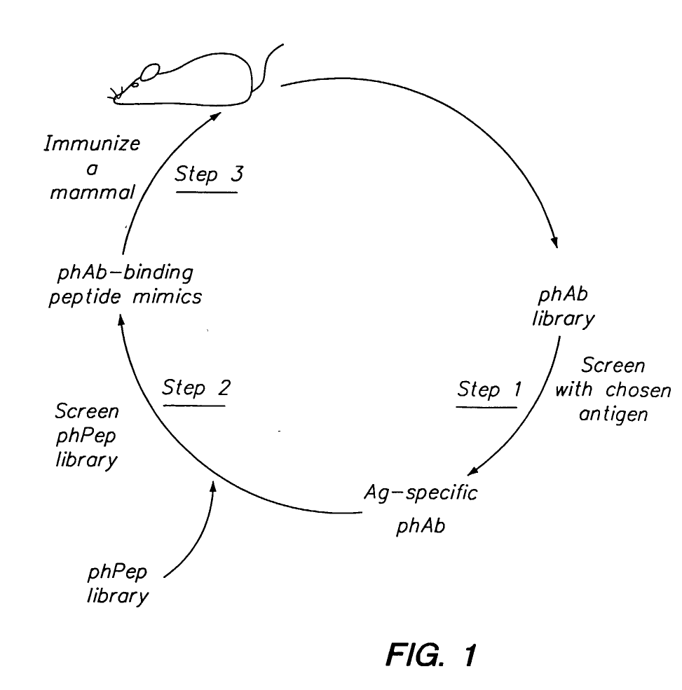

The instant invention presents, in a first aspect, a method for biasing the immune response of a mammal toward a desired epitope of a chosen antigen. FIG. 1 schematizes one embodiment of this method.

In the first step of the method for biasing the immune response, at least one phage-displayed antibody (phAb) is selected from a phage-displayed antibody library for its ability to bind to a chosen antigen.

This first step presupposes, of course, the existence of an appropriate phage-displayed antibody library, and FIG. 1 thus indicates construction of the library from a mouse. De novo construction of such a library is not required, however, if an appropriate library is otherwise available, and it is an object of the present invention to provide, for subsequent screenings, stored aliquots phage-displayed antibody libraries that have already been biased toward chosen antigens, either by prior immunization of the donor animal with the chosen antigen, or by the method described here, or by an interative alternation of the two.

The technology of phage-displayed antibodies is by now well-established, Rader et al., Current Opinion in Biotechnology 8:503-508 (1997); Aujame et al., Human Antibodies 8:155-168 (1997); Hoogenboom, Trends in Biotechnol. 15:62-70 (1997); de Kruif et al., 17:453-455 (1996); Barbas et al., Trends in Biotechnol. 14:230-234 (1996); Winter et al., Ann. Rev. Immunol. 433-455 (1994), and techniques and protocols required to generate, propagate, screen (pan), and use the antibody fragments from such libraries have recently been compiled, Phage Display of Peptides and Proteins: A Laboratory Manual, Kay, B K, Winter, J, McCafferty, J. (eds.), San Diego: Academic Press, Inc. 1996 (hereinafter, “Phage Display Manual”); Abelson et al. (eds.), Combinatorial Chemistry, Methods in Enzymology vol. 267, Academic Press (May 1996). The basic details of library construction, screening and expression need not, therefore, be repeated here, as they are, well within the knowledge of the skilled molecular biologist.

In addition, commercial kits are now available that allow the construction, propagation, and screening of phage display antibody libraries. Among these is the Recombinant Phage Antibody System (RPAS) available from Pharmacia Biotech (Amersham Pharmacia Biotech, catalogue number 27-9400-01), which proves particularly useful in the present invention. The RPAS system allows the expression of scFvs either as fusions to the pIII protein of filamentous phage for screening and propagation, or as soluble scFv antibody fragments for purposes of protein production. The form of the antibody fragment is determined by the choice of the chosen E. coli host strain. In addition, the RPAS system expresses the scFvs in tandem with an expression “tag” (“E” “tag”) which can be used for affinity purification or ELISA detection of the soluble scFvs.

Although not so indicated in FIG. 1, in preferred embodiments of the present invention the phage-displayed antibody library is constructed from mRNA derived from a human antibody-transgenic mouse, such as a Xenomouse™. In such case, the mRNA derived from the human antibody-transgenic mouse must be amplified with primers specific to human, rather than to mouse, immunoglobulin, prior to cloning into the display vector. Appropriate human primers are described in Marks et al., J. Mol. Biol. 222:581-597 (1991), and may be substituted for the primers provided in the RPAS kit.

In certain circumstances, it may be desired to increase the representation of variable regions found on IgG transcripts, thus increasing the proportion of variable regions that have undergone in vivo affinity maturation. It would be understood that such a strategy is best utilized in constructing libraries from animals that have previously been immunized with the chosen antigen and/or with an appropriate mimotope, as further described below.

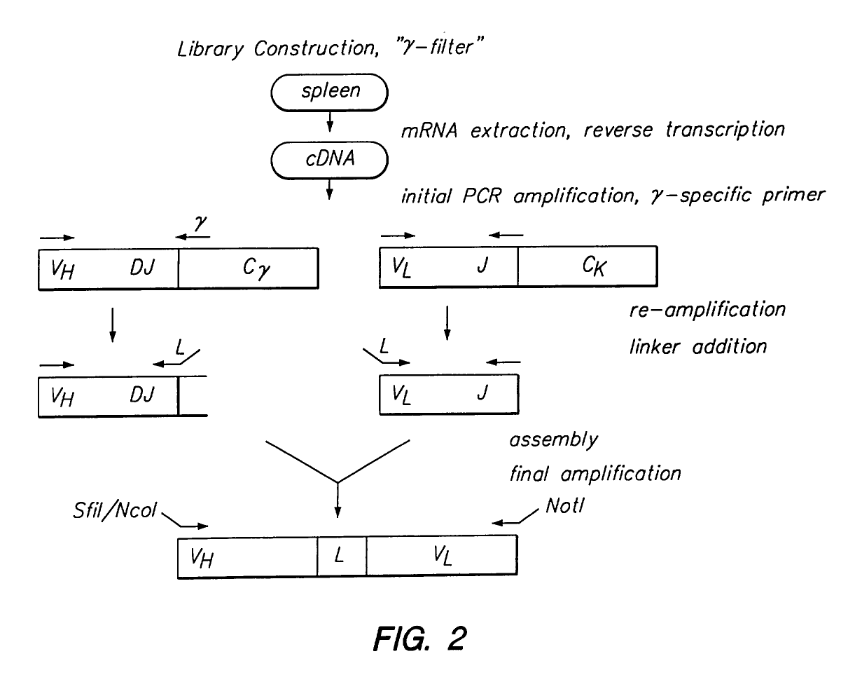

As shown in FIG. 2, such gamma-filtered libraries are constructed by using, in a first amplification step, a 3′ heavy chain primer that includes Cγ sequence, thus preferentially amplifying heavy chain variable regions found on gamma transcripts. A second amplification then permits the concurrent removal of the Cγ sequence from the amplified heavy chain products and the directional introduction of linkers to the 3′ end of V_{H} and the 5′ end of Vκ; this strategy permits assembly of the scFv fragment into the vector in a two-fragment, rather than 3-fragment process. The two-fragment assembly, as opposed to the three-fragment assembly directed by the RPAS kit and by Marks et al., lead to a significant enhancement in yield at the final assembly step.

The phAb library is screened with a chosen antigen to identify, with selected stringency, a polyclonal assortment of phAbs that bind to the chosen antigen. Although purified antigen may be used, more typically complex mixtures of antigen will be used, including whole cells or even tissue.

For example, a phAb library may be constructed from a Xenomouse™ immunized with a human melanoma cell line, and then screened (panned) to identify phAbs that bind to melanoma biopsy tissue from an individual patient. As is well known in the art, iterative pannings may be performed to increase the specificity of the resultant phage. In each such panning, the phage that are adsorbed to the selecting antigen are eluted, propagated by infection of male E. coli, and the selected and amplified phage then purified and again placed into contact with the selecting antigen. Typically, three to four such pannings are performed as part of this first screening step.

In addition, as is well known in the art, the specificity of the selected phage for the selecting antigen may be increased by first subtracting the library by adsorption to unrelated antigens. For example, the melanoma cell specificity of the phAbs selected on a melanoma biopsy may be increased by prior adsorption of the phAb library to related cell types, such as other neural crest derivatives, or to cell types likely found concurrently in the biopsy material, such as fibroblasts, keratinocytes, endothelial cells, and the like.

What results from this first screening step is a polyclonal mixture of phAbs that recognize different epitopes of the selecting antigen, or, in cases in which a mixture of antigens, such as whole cell or a tissue comprising multiple cells, is used to screen, a polyclonal mixture of phAbs that recognize multiple epitopes of a plurality of different antigens.

For example, the phAbs from a melanoma-cell biased immune library screened with a melanoma biopsy will contain phAbs specific for various immunodominant epitopes from the gp100 melanoma-associated antigen, Rosenberg et al., Nature Med. 4:321-327 (1998), phAbs specific for nonimmunodominant epitopes of the gp100 antigen, and phAbs specific for other immunodominant and nonimmunodominant antigens displayed in the melanoma biopsy.

As schematized in FIG. 1, the antigen-selected phAbs are then used in the second step of the method directly to screen a phage-displayed random peptide (PhPep) library.

In peptide phage display libraries, random peptides of defined length are cloned as fusions to either the gene III protein (pIII) or gene VIII protein (pVIII) for display on the surface of filamentous phage. Smith, Science 228:1315-1317 (1985); Scott et al., Science 249:386 (1990); Clackson et al., TIBS 12:173-184 (1994); Kay et al., Gene 128:59-65 (1993). The effective valency of the displayed peptide is determined in the first instance by the choice of protein fusion—pVIII is the major coat protein and pIII is the minor coat protein—and may further be manipulated by supplying a copy of the wild type gene, either on the same vector or on a phagemid. Bonnycastle et al., J. Mol. Biol. 258:747-762 (1996).

Because much of the technology is the same as that used in phage display of antibody fragments, protocols for generating, propagating, and screening such libraries may be found compiled in the Phage Display Manual, supra, and need not be further described here.

In addition, a single comprehensive peptide library, once constructed, may repeatedly be sampled; as a result, de novo construction of such libraries is not required, and commercial peptide epitope libraries may be purchased for such screening. New England Biolabs (Beverley, Mass.), for example, makes available for screening several random peptide libraries constructed in M13, with reagents necessary to screen the libraries (“Ph.D. phage display peptide libraries,” catalogue numbers 8100, 8110, 8210, and 8101). Each of the libraries is of high complexity, that is, includes greater than 109 independent clones, and has been used successfully to identify peptide ligands for several proteins, including antibodies. One of these libraries is a linear 7-mer library, one is a linear 12-mer library, and the last is a Cys-Cys constrained 7-mer library. As is well known in the art, each type of library presents certain advantages, and thus screening (panning) of a plurality of libraries, each with different construction, is often advisable. Rudolf et al., J. Immunol. 160:3315-3321 (1998).

Another commercial random peptide phage display library positions the random peptide instead in a flagella (Fli) thioredoxin (Trx) fusion protein, rather than on M13 gene III protein, as described in Lu, Bio/Technology 13:366-372 (1995) and U.S. Pat. No. 5,635,182, and is available commercially from Invitrogen (Carlsbad, Calif.; catalogue number K1125-01).

This second step of the biasing method identifies phage that bear peptides (“phPep”) that bind to the antigen-selected phAbs, mimicking epitopes of the original antigen (“mimotopes”). As in screening the phAb library, multiple rounds of selection increase the specificity at this step.

Typically, panning peptide libraries with an antibody will produce phage bearing several different peptide sequences. Alignment of these sequences will often result in a consensus sequence. In cases where this consensus sequence closely matches a continuous segment of the original antigen sequence, that is, mimics a linear epitope, it is possible to determine with some degree of certainty where the antibody binds on the antigen structure.

However, it is often the case that there is no recognizable alignment between the consensus sequence and the amino acid sequence of the antigen. In this latter case, the consensus sequence peptide may be assuming a conformation that mimics a conformational epitope of the original antigen. Alternatively, the consensus sequence may be mimicking a carbohydrate epitope on the antigen. In a further alternative, different parts of the consensus peptide sequence may be similar to physically distinct sequences on the native antigen, the peptide as a whole thus mimicking a discontinuous epitope on the antigen.

To confirm that a derived consensus sequence does, in fact, mimic a structure on the original antigen, a peptide of the consensus sequence may be synthesized chemically and used to confirm, first, that the consensus peptide binds to the panning (selecting) antibody, in this case, one or more antigen-selected phAbs, and second, that the consensus peptide competitively inhibits binding of the antibody to the selecting antigen. If both these criteria are met, it can be concluded that the consensus peptide is indeed a “mimotope” of a conformational determinant on the antigen.

Where several phAbs are used to screen the peptide library, additional complexity is added. For example, screening the phage-displayed random peptide library with the polyclonal assortment of phAbs that bind to a melanoma biopsy, as above-described, will produce peptides that mimic immunodominant epitopes of the gp100 melanoma-associated antigen, nonimmunodominant epitopes of the gp100 antigen, and epitopes of other antigens displayed in the melanoma biopsy.

As shown in FIG. 1, the peptide mimics selected in the second step are then used, in a third and final step, to immunize a mammal, thereby focussing the mammal's immune response on these identified epitopes, biasing the immune response toward such epitopes.

Although only a single mouse is shown in FIG. 1 as both donor of the phAb library and recipient of the mimotope immunization, it will be understood that where the donor mammal is sacrificed to construct the phAb library, a separate individual mammal must be immunized in this third step with the mimotopes.

The proper timing, dosage, and formulation of the peptide immunization are readily established by those skilled in antibody production.

The peptide display phage selected in the second step of the method may, for example, be used directly to immunize the animal, either alone, or after denaturation and admixture with adjuvant, such as complete or incomplete Freund's adjuvant.

A preferred approach, however, is to synthesize the encoded peptide mimics, or a consensus thereof, chemically, typically using a commercially available automated sGlid-phase peptide synthesizer.

The chemically-synthesized peptides, either collectively or individually, are then typically conjugated, using methods well known in the art, to a soluble protein carrier, such as KLH, BSA, or bovine thyroglobulin. Typical bifunctional conjugating reagents include m-maleimidobenzoyl N-hydroxysuccinimide ester (“MBS”), succinimidyl 4-(N-maleimido-methyl)-cyclohexane-1-carboxylate (“SMCC”), and 1-ethyl-3-(3-dimethylaminopropyl) carbodiimide (“EDAC”). Even glutaraldehye may be so used.

A particularly preferred alternative, however, to the serial steps of synthesis and conjugation of the peptide mimics to protein carriers, is to use the multiple antigen peptide procedure, Tam, Proc. Natl. Acad. Sci. USA 85:5409-5413 (1988); Tam et al., J. Immunol. Methods 124:53-61 (1989); Posnett et al., Methods Enzymol. 178:739-746 (1989), in which peptide synthesis is performed directly on a synthetic polylysine carrier. This system has advantages over the use of complex protein carriers in that the antibody response to the polylysine core is typically low, and the bulk of the antibodies are thus directed toward the conjugated peptide.

Another alternative is to immunize with a chemically-synthesized or recombinantly produced fusion protein, in which the peptide mimic is fused to a T cell epitope, Steward et al., J. Virol. 69:7668-7673 (1995), or to another polypeptide carrier. Yet another is to immunize with a synthetic or recombinantly produced peptide in which multiple copies of the peptide mimic are present. And still another alternative is to immunize not with conjugated peptide, but with unconjugated peptide, which has been shown to function adequately as an immunogen in certain circumstances. Atassi et al., Crit. Rev. Immunol. 5:387-409 (1985).

Still another alternative is to immunize not with peptide or protein, but with the nucleic acid encoding the peptide. It has now been shown in a number of systems that direct injection of nucleic acid can effectively immunize against the encoded product. U.S. Pat. Nos. 5,589,466 and 5,593,972; Hedley et al., Nature Med. 4:365-368 (1998); Ho et al., Arch. Virol. 143:115-125 (1998); Cardoso et al., J. Virol. 72:2516-2518 (1998); Bagarazzi et al., Curr. Top. Microbiol. Immunol. 226:107-143 (1998); Lozes et al., Vaccine 15:830-833 (1997); Shiver et al., Vaccine 15:884-887 (1997).

It will be understood that the above-described immunization with peptide mimics, whether accomplished by immunization with peptides displayed on phage, with synthetic peptides conjugated to carrier, or with nucleic acid, is not limited to a single injection, but may encompass immunization schedules that include both a primary and subsequent booster immunizations, with and without adjuvants, as is well understood in the immunologic arts.

In addition, the peptide immunizations may be alternated with immunization with whole antigen. Thus, the original phage-displayed antibody library may be derived from an animal first immunized with whole antigen, and the later-selected peptide mimics may be used to immunize a second animal that is either subsequently or antecedently immunized with whole antigen.

The result of this three-step method is to impose, upon a mammalian immune system, a bias toward the epitopes mimicked by the phage-displayed peptides.

As intimated by FIG. 1, the process may be reiterated, further biasing the immune response to desired epitopes of a chosen antigen. A second phage-displayed antibody library is constructed from the immunoglobulin transcripts of the peptide-immunized mammal; repeating the three steps above-described, this library is screened with a chosen antigen to identify antigen-specific phAbs, which, in turn, are used to screen a random peptide library, which, in a final step, are used to immunize yet another animal.

The result of this iterative method is a graduated series of phAb libraries with ever-increasing bias in favor of epitopes displayed by the desired antigen. These libraries are collectively termed “epitope-biased immune libraries” herein.

As mentioned above, an antigen will produce in the first step of this method, whether practiced singly or reiteratively, a polyclonal assortment of phAbs specific for a plurality of epitopes. This is especially true if selection of phAbs is conducted with a complex antigen, such as a mammalian cell line.

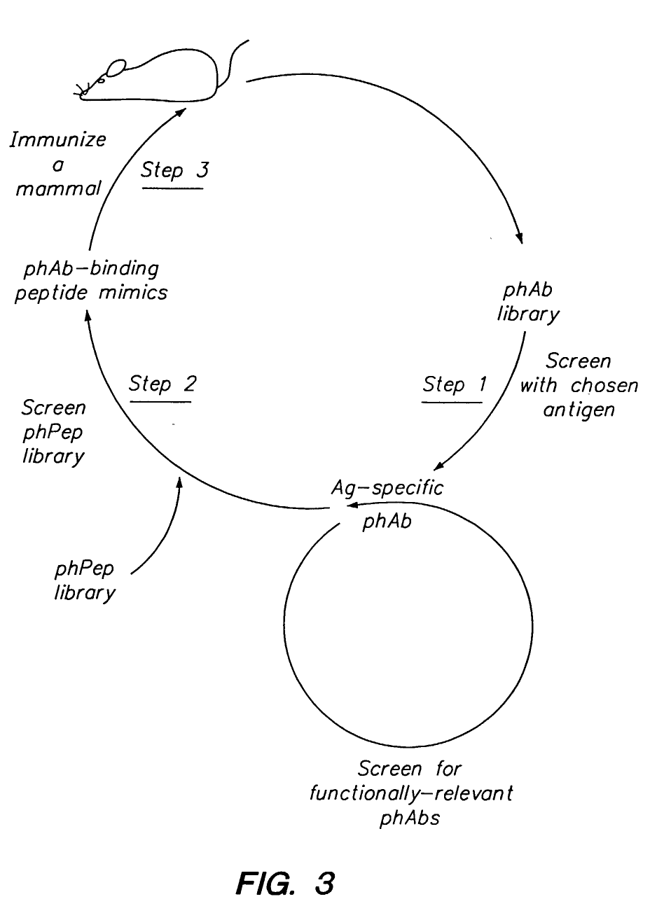

In a particularly preferred embodiment of the method, therefore, an additional step is interposed between screening the phAb library and screening the phPep library, as shown in FIG. 3. phAbs that bind to the chosen antigen are collected, amplified, and then subjected to a functional assay. Only those phAbs that functionally affect the antigen are used to screen the peptide library, thus biasing the immune response, in step 3, toward a desired functional epitope of a chosen antigen.

The assay interposed between library screenings is so chosen as to identify functionally-relevant epitopes, that is, antagonists of the chosen antigen, agonists thereof, or competitive inhibitors of ligands of the antigen; the choice of assay is dictated by the antigen and the desired functional result.

For example, in a method to bias the immune response to functionally-relevant and clinically-relevant epitopes of a melanoma cell, the phage-displayed antibodies selected upon a melanoma biopsy may be injected directly into a laboratory animal, as described in Pasqualini et al., Nature 380:364-366 (1996); Arap et al., Science 279:377-380 (1998); U.S. Pat. No. 5,622,699. If the mouse, typically a nude mouse, has previously been injected with a human malignant melanoma cell line, that subset of selected phage that homes to metastatic deposits, for example those in the mouse brain, may then be obtained by elution from such metastatic deposits and amplified. The phAbs so selected recognize epitopes displayed preferentially on metastatic cells.

Analogously, in a method to bias the immune response to clinically-relevant epitopes of L-selectin, phAbs that bind to L-selectin, as expressed on the surface of a human lymphoma cell line, may be further screened for their ability to inhibit the binding of lymphocytes to endothelial venules, and for their ability to discriminate cell-bound from cell-free L-selectin, as further disclosed in Example 1, below.

Furthermore, if one or more immunodominant epitopes of the antigen are known, but antibodies thereto are not desired, the functional screen may consist of a subtractive adsorption to peptides bearing the immunodominant epitope.

These antigenically-selected and functionally-selected phAbs are then used, in a second library screening, to identify peptide mimics of the epitopes recognized by these phAbs. The peptide mimics, in turn, are used in a final step as immunogens, in order to bias a mammal's immune response toward those epitopes.

Although the methods herein described have heretofore been discussed as using phage display libraries—both phage display antibody libraries and phage display random peptide libraries—it is intended and will be understood that comparable combinatorial display technologies, as now developed or as will be developed, may be adapted for use in these novel methods. Among such technologies are ribosome display, Hanes et al., Proc. Natl. Acad. Sci. USA 94:4937-4942 (1997) and retroviral display, Russell et al., Nucl. Acids Res. 21:1081-1085 (1993). Typically, these technologies will first be adapted to the display of random peptides, then later to the display of antibody genes.

The biased immune system of mammals that have been treated by the above-described method may then be surveyed, by either hybridoma or phage display technology, for specific high affinity immune reagents to desired epitopes of chosen antigens. Where the mammal is a human antibody-transgenic mammal, such as a Xenomouse™, the epitope-biased immune system may be sampled to generate high affinity human antibody reagents specific to a desired epitope of a chosen antigen, immediately suitable for in vivo use.

In one type of in vivo use, the identified epitopes may be targeted by human antibodies. The antibodies may be generated from the epitope-biased human transgenic mammal by standard hybridoma methods. Alternatively, phage displayed Fab or scFv fragments—either earlier chosen during the biasing itself, or newly constructed from the biased mouse—may be used. In yet another alternative, the binding moiety of such phage displayed antibodies may be cloned, using standard techniques, into vectors that direct expression of complete heterodimeric immunoglobulin chains or desired fusion proteins.

For example, Fab or scFv fragments from phage in a third iteration human melanoma epitope-selected library may be used in vivo to target diagnostic or therapeutic agents to melanoma cells. Although it is understood that the Fab or scFv identified in a combinatorial phAb library may not reproduce the heavy and light chain combinations that naturally occurs in the human (i.e., antibody-transgenic mouse) immune system, nonetheless the presence of exclusively human elements should prevent a host anti-Ig response.

Alternatively, the epitopes mimicked by the phage-displayed peptides produced in this method may themselves be used to induce an immune response in a human patient. For example, epitopes identified through the iterative selection of phAbs and phPeps on a melanoma biopsy may be prepared in suitable format and used to immunize a melanoma patient, either as individual peptides, as a consensus of such peptide sequences, or in combination, for induction of an active immune response in a patient against his own tumor. Rosenberg et al., Nature Med. 4:321-327 (1998).

It will also be appreciated that the epitopes to which the iteratively selected epitope-biased immune libraries are biased include epitopes that are not recognized by the mouse immune system, and thus include epitopes that have not previously been used in diagnostic or therapeutic methods.

Alternatively, an entire repertoire of antibodies or phAbs from the immunized animal may be created, either to serve as a library to be sampled in subsequent iterations of the above-described method, or to provide an epitope-biased immune library for determination of epitope expression profiles, as will now be described.

The methods described hereinabove permit the identification of functional epitopes of chosen antigens and the generation of specific immune reagents thereto. Thus, for antigens suspected to be clinically relevant, the method provides a direct route to reagents—including fully human antibodies of subnanomolar affinity—that functionally affect such chosen targets.

On occasion, however, the antecedent question arises whether a particular protein presents such clinically-relevant antigens. With the accelerating pace with which new genes are being identified, and identified solely by nucleic acid sequence data, the question increasingly is raised as to the biologic, physiologic, and clinical relevance of a newly discovered gene's expression product.

It is, therefore, a further object of the present invention to provide compositions, methods, and apparatuses for determining epitope expression profiles of genomics-derived genes. As used herein, the phrase “epitope expression profile” denotes a data set, specific for a given protein, each data point of which reports a measure of the binding of the protein to a library of antibodies. Where the antibody libraries are variously biased—as, for example, toward distinct tissues or cell types—the epitope expression profile provides a topography of the biologic availability of the protein's epitopes in the tissues and cell types so surveyed.

Thus, a first step in the creation of such profiles is the generation of immune libraries biased to distinct tissues and cell types. In preferred embodiments, these libraries are constructed from human antibody-transgenic mice, thus providing libraries of fully-human antibodies.

To create a biased library, mice, preferably human antibody-transgenic mice, are appropriately immunized with a chosen tissue or cell line. Table 1 lists tissue immunogens that are useful in the present invention. It should readily be appreciated that this listing is neither comprehensive nor limiting, but serves instead to identify an initial sampling of tissues that are particularly useful in the creation of biased libraries for the further construction of epitope expression profiles.

TABLE 1
Tissue Immunogens

| | |
|---|---|
| adipose tissue | heart |
| adrenal | kidney |
| aorta | liver |
| bone marrow | lung |
| brain (whole) | lymph node |
| brain (amygdala) | ovary |
| brain (cerebellum) | pancreas |
| brain (hippocampus) | pituitary |
| brain (substantia nigra) | prostate |
| brain (corpus striatum) | eye (whole) |
| brain (hypothalamus) | eye (retina) |
| brain (subthalamic | skeletal muscle |
| nucleus) | |
| brain (frontal cortex) | small intestine |
| brain (occipital cortex) | spinal cord |
| brain (temporal cortex) | spleen |
| breast | stomach |
| colon | testis (whole) |
| cornea | testis (epididymis) |
| placenta | thymus |
| skin | uterus |
| synovial membrane | myelin |

Cell lines, particularly human cell lines, also prove particularly useful in the generation of biased libraries for production of epitope expression profiles. Many such cell lines, representing immortalized but untransformed cells, neoplastically transformed cells, and virally-immortalized cells, are available from the American Type Culture Collection (ATCC); others, carrying defined genetic mutations, are available from the National Institute of General Medical Sciences' Human Genetic Mutant Cell Repository, housed at the Coriell Institute for Medical Research of the University of Medicine and Dentistry of N.J. (Camden, N.J.).

Cell lines are particularly useful and important in biasing libraries to neoplastic cells, as many existing cell lines are neoplastically transformed. Among the neoplastically transformed cell lines useful in the present invention are colorectal carcinoma cell lines, prostate carcinoma cell lines, renal carcinoma cell lines, melanoma cell lines, breast carcinoma cell lines, lung carcinoma lines, lymphoma and leukemia lines, erythroleukemia cell lines, glioma cell lines, neuroblastoma cell lines, sarcoma including osteosarcoma cell lines, hepatocellular carcinoma cell lines, and the like.

Immortalized, yet untransformed cell lines that are preferably used include, but are not limited to, B cell lines at various stages of differentiation, T cell lines at various stages of differentiation, neutrophil cell lines, NK cell lines, macrophage cell lines, megakaryocytic cell lines, monocyte cell lines, dendritic cell lines, and the like.

Furthermore, biased libraries may be constructed from normeoplastic cells and tissues that are infected with virus, such as HIV, HBV, human herpesviruses, HCV, bacteria including mycobacteria, or eukaryotic pathogens such as trypanosomes. In addition, tissues that are involved in ongoing autoimmune processes, such as synovial membranes from patients with rheumatoid arthritis, may also be used.

Furthermore, it will be readily apparent that further distinctions and finer discrimination may be made, with additional libraries generated to distinguishable subcellular fractions derived from the aforementioned tissues and cells.

After immunization, antibody libraries are created using either hybridoma or phage display techniques. Because the latter technology is described in detail above, the following discussion will focus on hybridoma libraries, although it should be understood that phage displayed antibody libraries are also useful in the present method.

With respect to hybridoma production, the procedures used for human antibody-transgenic mice are substantially identical to those used for standard nontransgenic mouse strains, as compiled in Delves et al., Antibody Production: Essential Techniques, John Wiley & Sons (1997); Lennox et al. (eds.), Monoclonal Antibodies: Principles and Applications, John Wiley & Sons (1995); Liddell et al., A Practical Guide to Monoclonal Antibodies, John Wiley & Sons (1991); and Harlow et al., Antibodies: A Laboratory Manual, Cold Spring Harbor Press (1988), and need not be described in detail.

Briefly, however, the immunized animal, or plurality of animals identically so immunized, is sacrificed, splenic lymphocytes harvested, and the lymphocytes fused to an immortal fusion partner, such as a nonproducing murine myeloma cells. After selective culture, hybridomas are disposed in microtiter dishes for further culture.

Each biased library thus is a polyclonal assortment of monoclonal antibody-producing hybridoma cells. Where the immunized animal is a human antibody-transgenic mouse, the hybridomas secrete human antibody. These hybridomas collectively reproduce the humoral immune response of the donor mouse. Some of the antibodies secreted by these hybridomas will be directed to epitopes uniquely displayed on the chosen immunogen, some of these with high affinity, including antibodies of subnanomolar affinity. Others will be specific to epitopes shared by the chosen immunogen and other cell types. Still others will be directed to antigens unrelated to those on the original immunogen. Each such collection of hybridoma cells, then, represents a library of antibody-producing cells, the collective repertoire of which is biased, as compared to a the nonimmunized reference mouse, in favor of the immunizing tissue or cell type.

Although these biased libraries may be used in the subject invention without further selection, the bias may be rendered more pronounced, and the collection of antibodies produced thus more specific for the original immunogen, by elimination of hybridomas that secrete antibodies recognizing shared or unrelated epitopes. Alternatively, the bias of the library may be rendered more pronounced by an antecedent step of tolerizing the mice to unrelated, or closely related, antigens.

For reference purposes, libraries are also prepared from unimmunized antibody-transgenic mice.

The hybridomas from each of the biased libraries—either directly from the fusion, or after further selection for immunogen-specific hybridoma clones—are then cloned into spatially-addressable matrices for storage and for assay.

For storage, the hybridomas may be cloned using standard techniques into separate, individually identifiable wells of tissue-culture microtiter dishes, and frozen.

For assay, three basic formats are preferred: (1) a “single-pot” library of antibodies disposed upon a BIACore® sensor; (2) a spatially-addressable matrix of antibody-secreting hybridomas, and (3) a spatially-addressable matrix of the antibodies themselves. The first and third formats are equally applicable to hybridoma-produced antibody libraries and phage-displayed antibody libraries. The first format is preferred, and use of the first format with phage-displayed antibody fragments is particularly preferred, with scFv fragments especially preferred.

The BIACore® measures binding of unlabeled ligands to surface-immobilized molecules using the optical phenomenon of surface plasmon resonance. The BIACore® has been used, inter alia, to monitor the affinity of phage-displayed antibodies. Schier et al., Hum. Antibod. Hybridomas 7:97-105 (1996); Schier et al., J. Mol. Biol. 255:28-43 (1996); Schier et al., J. Mol. Biol. 263:551-567.

In the present application, the antibodies from a minimally-amplified biased library are themselves immobilized on the BIACore® sensor chip using techniques well known in the art and well described in Malmborg et al., J. Immunol. Methods 183:7-13 (1995); Wong et al., J. Immunol. Methods 209:1-15 (1997); and in the BIACore® product literature. Each sensor chip can contain an entire biased antibody library, and may repeatedly be assayed.

In contrast to the two other formats further described below, the single-pot BIACore® format does not dispose the antibodies in a spatially-addressable format. Instead, the antibodies from an entire library are disposed at random, and the BIACore® reports an aggregate level of binding of the polypeptide ligand thereto.

With respect to the second of the three formats—a spatially-addressable matrix of antibody-secreting hybridomas—the matrix will typically be constructed in standard tissue culture-compatible microtiter plates. A biased immune library will occupy a plurality of such plates, with the number inversely related to the stringency of the post-fusion selection for immunogen specificity. One advantage of using standard microtiter dishes for assay is the ready availability of robotic devices specifically designed to manipulate the contents of such plates.

In a third alternative format, the library may be constructed without cellular components, using either the hybridoma supernatants, purified fractions thereof, in either liquid or solid phase, or phage-displayed antibodies.

In this last typical format, as with the hybridoma matrix, supernatants and purified antibodies in either liquid or dry form may be arrayed in standard microtiter plates, to similar advantage. Other geometries, however, prove uniquely advantageous with noncellular matrices; in particular, the antibodies may be immobilized, substantially free of aqueous media, in spatially addressable matrices or linear arrays on solid supports, such as those typically used in the immunoassay arts.

Each single-pot BIACore® sensor chip or each spatially-addressable surface-immobilized antibody matrix represents the collective antibody response of a biased immune library; each presents a distinctive collection of antibodies with specificity for antigens that are expressed on normal, mutant, or diseased tissues and cells. These surface-immobilized antibody libraries may then be used to screen the expression products of any identified open reading frame to determine the tissue-specific or cell-type specific pattern of its epitopic availability.

The first assay format, in which the antibodies or antibody fragments are disposed upon a BIACore® sensor chip, does not require a label for detection of the binding of the gene expression product to the antibody library. The other two assay formats require a label.

Although several labeling and detection formats common in the immunoassay art may be used—as reviewed most recently in Diamandis et al. (eds.), Immunoassay, Amer. Assn for Clinical Chemistry (1997); Price et al. (eds.), Principles and Practice of Immunoassay, Stockton Press (1997); Deshpande, Enzyme Immunoassays: from Concept to Product Development, Chapman & Hall (1996); and Chan (ed.), Immunoassay Automation: An Updated Guide To Systems, Academic Press (1996)—a geometry that is particularly well-adapted to the multiple use of any given library leaves the

Each of these known algorithms may be adapted to comparison of epitope expression profiles, to identify, for any gene, the cell- and tissue-specific expression of its epitopes.

An important advantage of epitope expression profiling, as above-described, over other technologies for measuring patterns of gene expression, is that epitope profiling provides a direct route to specific antibodies for further research or clinical investigation: every element of an immobilized biased library that returns a positive signal for a given gene's expression product, represents an antibody that necessarily recognizes the protein. These antibodies, as so identified during assay, may then be used individually, free of the support matrix, further to define the expression pattern and function of the gene of interest.

The identified antibodies can be used as research reagents for evaluation of protein function. Since the antibodies are, in preferred embodiments, fully human, they can serve as lead candidates for in vivo assays, and potentially, for in vivo therapeutic or diagnostic use. Furthermore, in the preferred embodiments using fully human antibodies, a different universe of epitopes from that which has now been exhaustively sampled through use of murine hybridoma technology may be identified.

An advantage of using phage-displayed biased libraries in the construction of immobilized libraries (either single-pot BIACore® libraries or spatially-addressable matrices), over libraries constructed using hybridomas, is the ready generation of libraries containing 10⁵-10¹⁰ discrete antibody elements (also termed binding nodes). Preferably, such matrices will include 10⁶-10¹⁰ binding nodes, more preferably 10-10¹⁰, most preferably 10⁸-1×10¹⁰. For hybridoma-based matrices or single-pot libraries, typically no more than 10³-10⁵ such binding nodes will be present, preferably 10⁴-10⁵, most preferably, from 5×10⁴ to 1×10⁵, although higher numbers remain possible and are always preferred.

A disadvantage of phage-displayed biased libraries in the construction of immobilized libraries, however, is the absence of complete heterodimeric fully-human antibodies corresponding to the elements that report a positive signal from the matrix (or single-pot BIACore® sensor chip. However, it is well within the skill in the art to use the identified binding moiety, particular phage-displayed Fab fragments, to reconstruct an intact, heterodimeric antibody using standard techniques. Such recombinant antibodies may then be expressed from anv of a number of mammalian cell types, including non-producing myeloma cells (e.g., NSO cells), hybridomas, chinese hamster ovary (CHO) cells, and the like. See, e.g., Page, U.S. Pat. Nos. 5,545,403, 5,545,404, 5,545,405; Page et al., Biotechnology 9:64-68 (1991); Peakman et al., Hum. Antibodies Hybridomas 5:65-74 (1994).

The following examples are offered by way of illustration and not by way of limitation.

### EXAMPLE 1

### Human Antibodies to Cell-Bound L-Selectin

Jurkat cells (ATCC catalogue number TIB-152) maintained in cell culture are concentrated by centrifugation, rinsed in PBS, and an aliquot of 10⁷ cells emulsified in complete Freund's adjuvant to a final volume of 100 μL.

Human antibody transgenic mice of the Xenomouse™ strain, Mendez et al., Nature Genetics 15:146-156 (1997), are injected with 100 μL of emulsified cells, either intraperitoneally or subcutaneously at the base of the tail, according to standard techniques, Delves et al., Antibody Production: Essential Techniques, John Wiley & Sons (1997); Harlow et al., Antibodies: A Laboratory Manual, Cold Spring Harbor Press (1988). Additional immunizations are performed using an equivalent number of Jurkat cells emulsified in incomplete Freund's adjuvant at two-week intervals for a total of 3-5 immunizations.

Within 2 weeks of the final immunization, the spleen is harvested from each Jurkat-immunized mouse, mRNA isolated by standard techniques, and the mRNA reversed transcribed into cDNA, using reagents and protocols packaged in the Pharmacia RPAS system.

Initial PCR amplification is performed, as shown in FIG. 2, with human primers, Marks et al., J. Mol. Biol. 222:581-597 (1991), with the 3′ heavy chain primer substituted with a primer complementary to a sequence common to all human IgG subclasses.

In a second PCR amplification, as shown in FIG. 2, the gamma sequence is eliminated and extended, overlapping, linker sequences are added to the 3′ end of V_{H} and the 5′ end of Vₖ. Thereafter, two-fragment PCR generates scFv fragments that are cloned into the SfiI and NotI sites in the pCANTAB 5E phagemid vector supplied with the Pharmacia RPAS Expression Module. The phagemids are then used to transform E. coli TGi cells, and phage rescue is performed by infection with M13K07 helper phage, in accord with the manufacturer's instructions.

Phage that bear scFvs that bind L-selectin are selected using the RPAS recombinant phage selection module with biotinylated L-selectin-IgG, essentially as provided in the kit instructions.

Selected phage clones that are reactive with L-selectin are used to infect E. coli HB2151 cells to induce secretion of scFvs into the medium. The SCFvs are purified using the Pharmacia RPAS purificaiton module, according to instructions.

The soluble scFvs are next assayed in three separate assays.

First, the scFvs are used in an ELISA to confirm binding to recombinant L-selectin-IgG fusion protein. Additional ELISAs are used to determine binding to nonchimeric, affinity-purified L-selectin isolated from human serum, Schleiffenbaum et al., J. Cell. Biol. 119:229-238 (1992), and to free IgG.

Second, scFvs that bind the L-selectin-IgG fusion protein but not IgG or free, soluble L-selectin are further tested in a functional assay for their ability to compete with anti-LAM1-1 for binding to selectin-IgG in a competitive ELISA. Anti-Lam1-1 is a murine antibody that blocks binding of L-selectin to endothelial cells and binds only to the surface-bound form. Schleiffenbaum et al., J. Cell. Biol. 119:229-238 (1992); Kansas et al., J. Cell. Biol. 114:351-358 (1991); Spertini et al., J. Immunol. 147:942-949 (1991).

Third, scFvs that bind L-selectin fusions but not shed L-selectin, and that further compete with anti-LAM1-1 for binding, are tested in a functional assay for inhibition of lymphocyte adhesion to endothelial cells. For this purpose, an in vitro Stamper-Woodruff frozen section assay is used, essentially as described in Stamper et al., J. Exp. Med. 144:828 (1991). Briefly, frozen sections of mouse peripheral lymph nodes are mounted on glass slides. These slides are then incubated for five minutes at 4° C. with 5×10⁶ 300.LAM1 cells (Tedder et al., J. Immunol. 144:532 (1990)), resuspended in 100 μL RPMI with 10% fetal calf serum (FCS), together with 100 μL of scFv.

Several scFvs that inhibit binding of 300.LAM1 cells are isolated, and their corresponding phage amplified in E. coli.

The phAbs so selected in the above three assays are then individually used to screen commercial phage-displayed random peptide libraries (New England Biolabs). Each of the NEB libraries is screened in parallel with each such phage-displayed scFv. The magnetic bead method of phage selection is used to screen the peptide libraries, as described in Harrison et al., Methods Enzymol. 267:83-109 (1996).

Briefly, 2.5 ml of peptide phage (approximately 10¹² titer units), 2.5 ml 4% MPBS, 50 μL Tween 20, and soluble scFv antibody are mixed together in a 15 ml tube and rotated at room temperature for 1 hour. In the first round of selection the concentration of scFv approximates 50 nM, which is reduced in subsequent rounds, as necessary, to select for higher affinity binding. Then 1.5 ml streptavidin Dynabeads coated with S,S-biotinylated anti-E Tag antibody (Pharmacia RPAS system) is then added to the phage-antibody mix and rotated for an additional 15 minutes. After three cycles of washing, twice with 1 ml PBS and once with 12 ml 2% MPBS, the phage are eluted with PBS containing 50 mM DTT. The eluted phage are then titered and repropagated in preparation for further rounds of selection, as set forth above. After four rounds of selection, individual clones are picked, propagated, and sequenced using primers provided by NEB for use with its phage-displayed peptide libraries.

The peptide sequences are input into a computer, translated and the amino acid sequences aligned to derive one or more consensus sequences. Each such consensus peptide is then synthesized as a fusion to a synthetic polylysine carrier according to Tam, Proc. Natl. Acad. Sci. USA 85:5409-5413 (1988); Tam et al., J. Immunol. Methods 124:53-61 (1989); Posnett et al., Methods Enzymol. 178:739-746 (1989).

Additionally, the following are synthesized on polylysine carriers: (1) several peptides with sequence exactly as displayed on the selected phage (phagotopes), among which is included the tightest binding phage, as determined by comparing all the phagotopes in a quantitiatve ELISA assay as described by Valadon et al., J. Immunol. Methods 197:171-179 (1996); (2) several peptides in which the sequence as displayed on the selected phage has been extended based on the sequence of human L-selectin; (3) several consensus peptides the sequence of which is extended based on the flanking-residues in the contributing sequences, per Barchan et al., J. Immunol. 155:4264-4269 (1995).

XenoMice are then immunized individually with one of the peptide conjugates using a standard repetitive immunization schedule. One half of the animals also receive alternative immunization with 300.LAM1 cells. Serum titers are periodically tested against both the peptide and L-selectin-IgG.

Animals displaying titers of anti-L-selectin-IgG antibodies in serum are sacrificed, their spleens harvested, and fused to create libraries of hybridomas, according to standard techniques.

In the first screen of the hybridoma supernatants, approximately two weeks post-fusion, the supernatants are tested in two parallel ELISA assays, one testing for binding of the mimotope conjugated to a different carrier (KLH, BSA, or bovine thyroglobulin), and one testing for binding to L-selectin-IgG fusion protein. Horseradish peroxidase (HRP)-conjugated goat anti-human IgG is used as a detection agent, as it does not cross react with murine IgG, so there is no risk of the detection agent binidng to the murine IgG moiety of the L-selectin chimeric fusion protein.

Hybridomas that test positive for binding to L-selectin are further tested for the presence of human kappa light chain, and for binding to serum-derived soluble L-selectin. Hybridomas that produce fully ability to compete with anti-LAM1-1 for binding to L-selectin-IgG in a competitive ELISA. Anti-Lam1-1 is a murine antibody that blocks binding of L-selectin to endothelial cells and binds only to the surface-bound form. Schleiffenbaum et al., J. Cell. Biol. 119:229-238 (1992); Kansas et al., J. Cell. Biol. 114:351-358 (1991); Spertini et al., J. Immunol. 147:942-949 (1991).

Third, scFvs that bind L-selectin fusions but not shed L-selectin, and that further compete with anti-LAM1-1 for binding, are tested in a functional assay for inhibition of lymphocyte adhesion to endothelial cells. For this purpose, an in vitro Stamper-Woodruff frozen section assay is used, essentially as described in Stamper et al., J. Exp. Med. 144:828 (1991). Briefly, frozen sections of mouse peripheral lymph nodes are mounted on glass slides. These slides are then incubated for five minutes at 4° C. with 5×10⁶ 300.LAM1 cells (Tedder et al., J. Immunol. 144:532 (1990)), resuspended in 100 μL RPMI with 10% fetal calf serum (FCS), together with 100 μL of scFv.

Several scFvs that inhibit binding of 300.LAM1 cells are isolated, and their corresponding phage amplified in E. coli.

The phAbs so selected in the above three assays are then individually used to screen commercial phage-displayed random peptide libraries (New England Biolabs). Each of the NEB libraries is screened in parallel with each such phage-displayed scFv. The magnetic bead method of phage selection is used to screen the peptide libraries, as described in Harrison et al., Methods Enzymol. 267:83-109 (1996).

Briefly, 2.5 ml of peptide phage (approximately 10¹² titer units), 2.5 ml 4% MPBS, 50 pL Tween 20, and soluble scFv antibody are mixed together in a 15 ml tube and rotated at room temperature for 1 hour. In the first round of selection the concentration of scFv approximates 50 nM, which is reduced in subsequent rounds, as necessary, to select for higher affinity binding. Then 1.5 ml streptavidin Dynabeads coated with S,S-biotinylated anti-E Tag antibody (Pharmacia RPAS system) is then added to the phage-antibody mix and rotated for an additional 15 minutes. After three cycles of washing, twice with 1 ml PBS and once with 12 ml 2% MPBS, the phage are eluted with PBS containing 50 mM DTT. The eluted phage are then titered and repropagated in preparation for further rounds of selection, as set forth above. After four rounds of selection, individual clones are picked, propagated, and sequenced using primers provided by NEB for use with its phage-displayed peptide libraries.

The peptide sequences are input into a computer, translated and the amino acid sequences aligned to derive one or more consensus sequences. Each such consensus peptide is then synthesized as a fusion to a synthetic polylysine carrier according to Tam, Proc. Natl. Acad. Sci. USA 85:5409-5413 (1988); Tam et al., J. Immunol. Methods 124:53-61 (1989); Posnett et al., Methods Enzymol. 178:739-746 (1989).

Additionally, the following are synthesized on polylysine carriers: (1) several peptides with sequence exactly as displayed on the selected phage (phagotopes), among which is included the tightest binding phage, as determined by comparing all the phagotopes in a quantitiatve ELISA assay as described by Valadon et al., J. Immunol. Methods 197:171-179 (1996); (2) several peptides in which the sequence as displayed on the selected phage has been extended based on the sequence of human L-selectin; (3) several consensus peptides the sequence of which is extended based on the flanking residues in the contributing sequences, per Barchan et al., J. Immunol. 155:4264-4269 (1995).

XenoMice are then immunized individually with one of the peptide conjugates using a standard repetitive immunization schedule. One half of the animals also receive alternative immunization with 300.LAM1 cells. Serum titers are periodically tested against both the peptide and L-selectin-IgG.

Animals displaying titers of anti-L-selectin-IgG antibodies in serum are sacrificed, their spleens harvested, and fused to create libraries of hybridomas, according to standard techniques.

In the first screen of the hybridoma supernatants, approximately two weeks post-fusion, the supernatants are tested in two parallel ELISA assays, one testing for binding of the mimotope conjugated to a different carrier (KLH, BSA, or bovine thyroglobulin), and one testing for binding to L-selectin-IgG fusion protein. Horseradish peroxidase (HRP)-conjugated goat anti-human IgG is used as a detection agent, as it does not cross react with murine IgG, so there is no risk of the detection agent binidng to the murine IgG moiety of the L-selectin chimeric fusion protein.

Hybridomas that test positive for binding to L-selectin are further tested for the presence of human kappa light chain, and for binding to serum-derived soluble L-selectin. Hybridomas that produce fully human antibodies and bind L-selectin IgG but not soluble L-selectin are subcloned. The subclones are expanded for production of antibody in the range of 100-500 mg in bioreactors. IgG is purified from the culture medium and quantified.

The hybridoma-produced heterodimeric fully human IgG molecules are then tested for their ability to inhibit lymphocyte binding in a Stamper-Woodruff assay, as described above. The quality of the antibodies is further assessed by measuring their affinity for L-selectin-IgG on the BIACore®.

Using this process for biasing the immune response of human antibody-transgenic mice toward functional epitopes of L-selectin, fully human IgG/κ antibodies are produced with the following properties.

First, the antibodies discriminate cell-bound from shed L-selectin, binding to L-selectin-IgG and L-selectin displayed on cell surfaces, but not to soluble L-selectin affinity purified from human serum. Second, the antibodies are able to inhibit lymphocyte binding to endothelial cells in the Stamper-Woodruff assay. Third, the antibodies have affinities that range from 10 nM (1×10⁻⁸M) to 50 pM (5×10⁻¹¹ M), with the majority of antibodies having affinities in the range of 1 nM to 100 pM. These antibodies are suitable for use as in vivo agents to abrogate immune responses that require the function of cell-bound L-selectin.

### EXAMPLE 2

### Generation of Antibodies Which are Selective for B7-1 and B7-2

In this example, the use of methods of the present invention arc discussed in the context of the generation of antibody candidates that bind to both the B7-1 and B7-2 molecules. Such molecules arc involved in B cell and T cell communication and stimulation. Molecules that act on one or the other, but not both, are not anticipated to be therapeutically valuable. Thus, there has been a considerable interest in generating a molecule that acts against both molecules.

A. Generation of Tissue Biased Library

Human antibody transgenic mammals are immunized with a B cell line to generate a “panel of antibody moieties” or a “tissue biased library” using conventional techniques. Such library can be presented as a panel of hybridoma cells, a panel of hybridoma supernatants, a panel of antibodies, a panel of phage, or otherwise. To generate the library, in general B cells are taken from the mouse and either fused to form hybridomas or subjected to molecular biological techniques, such as RT-PCR, to pull out cDNAs to form display libraries. Once the library is established, it will be understood that it will contain variable region sequences that have been biased towards the recognition of the antigens and epitopes displayed on the B cells used for immunization of the mammal.

B Screening of the Tissue Biased Library

The panel or library is screened or probed against the target molecule, either B7-1 or B7-2 in the first instance. Antibody moities that bind to the target molecule, and particularly those that bind with an affinity of greater than or equal to 10⁸ M arc selected for continued study. Binding and affinity can be measured using conventional techniques such as ELISA and BIACore for example

C. Functional Assessment of the Selected Antibody Moieties

Those antibody molecules that are selected in B above are next assessed for their desired function. In the present example, cross reactivity of the antibody moieties with B7-1 and B7-2 would be assessed. Further, an assay in which B cells cultured with T cells in the presence of an anti-CD3 antibody could be utilized to determine if the antibody moieties inhibited the production of IL-2 in the culture. IL-2 production is dependent upon binding of B7-1 and/or B7-2 to the counter-receptor, CD28 on T-cells. Those antibody moieties that were cross reactive with B7-1 and B7-2 and inhibited IL-2 production in the above assay would be selected for further study.

The process of selection of antibody candidates could be terminated at this stage since candidates that possess the desired function have been identified. However, it is possible to generate additional antibody candidates with similar function and enhanced binding through conducting additional steps in accordance with the present invention. Indeed, since the goal of the present invention is the generation of therapeutic candidates, it is desirable to have numerous antibodies with the desired characteristics for evaluation.

D. Screening Antibody Moieties for the Selection of Mimotopes

As discussed in Example 1, the antibody candidates identified above can be screened against peptides or other epitopic determinants to identify mimotopes of the epitopes to which the selected antibody candidates bind. Such screening can be accomplished using conventional techniques that are well known in the art.

E. Immunization of a Human Antibody Transgenic Mammal with Selected Mimotopes and Selection of Antibodies

Mimotopes selected above are next utilized to immunize human antibody transgenic mammals to generate a specific immune response against the epitopic determinant present on the mimotope. B cells are harvested and generally fused using conventional techniques to generate hybridoma cell lines. Such hybridoma cells lines, or supernatants or antibodies obtained therefrom, are generally screened against mimotope and the antigens of interest (here, cross-reactivity with B7-1 and B7-2 and blocking binding of B7-1 and B7-2 to CD28) and assessed for binding affinity (i.e. generally greater than 10⁻⁸).

As will be appreciated, the same approach as delineated above can be used in connection with the generation of antibody moieties to a target molecule of “unknown” or incompletely characterized function. This is particularly useful in connection with the generation of early therapeutic leads for genomics type target molecules. This is to say that once a target molecule is identified and sufficient functional information about the target molecule is known to establish functional assays, the methods of the present invention can be utilized to rapidly generate high affinity human monoclonal antibodies that specifically bind to the target molecule and possess certain desired functions as determined by the functional assays.

It will be appreciated that the present invention is not limited to extracellular targets. Indeed, the methods of the present invention are also useful in connection with the generation of intrabodies which may prove useful in connection with acting as antagonists or agonists to intracellular targets.

All patents, patent publications, and other published references mentioned herein are hereby incorporated by reference in their entirety as if each had been individually and specifically incorporated by reference herein.

While a preferred illustrative embodiment of the present invention is described, it will be apparent to one skilled in the art that various changes and modifications may be made therein without departing from the invention, and it is intended in the appended claims to cover all such changes and modifications which fall within the true spirit and scope of the invention.

What is claimed is:

1. A method of biasing the immune response of a mammal toward a desired epitope of a chosen antigen, comprising the steps of:
(a) selecting, from a phage-displayed antibody library, at least one phage-displayed antibody (phAb) that binds to said antigen; then
(b) selecting, from a phage-displayed peptide library, at least one phage-displayed peptide that binds to said antigen-specific phAb and that mimics a desired epitope of said antigen; and then
(c) immunizing a mammal with said peptide mimic.

2. The method of claim 1, further comprising at least one iteration of the subsequent steps of:
(d) constructing a phage-displayed antibody library from immunoglobulin transcripts of said peptide mimic-immunized mammal; followed in order by steps (a)-(c).

3. The method of claim 1, further comprising the step, after step (b) or after step (c), of:
immunizing said mammal with said antigen.

4. The method of claim 1, further comprising the step, after step (a) and before step (b), of:
further selecting from the phAbs selected in step (a), for further use in step (b), only those phAbs that functionally affect said antigen.

5. The method of claim 1, wherein said phage-displayed antibody library is constructed from an antibody-transgenic mammal.

6. The method of claim 5, wherein said antibody-transgenic mammal is a human antibody-transgenic mammal.

7. The method of claim 6, wherein said antibody-transgenic mammal is a mouse.

8. The method of claim 1, wherein said phage-displayed antibody library preferentially includes variable regions derived from IgG transcripts.

9. The method of claim 1, wherein said phage-displayed peptide mimics are selected in step (b) by screening said phage-displayed peptide library with at least one of said antigen-specific phAbs.

10. The method of claim 1, wherein, in step (c), said immunizing peptide mimic is a phage-displayed peptide selected in step (b).

11. The method of claim 1, wherein, in step (c), said immunizing peptide mimic is chemically-synthesized.

12. The method of claim 11, wherein said chemically-synthesized peptide includes the amino acid sequence of a phage-displayed peptide selected in step (b).

13. The method of claim 11, wherein said chemically-synthesized peptide includes an amino acid sequence that is a consensus of amino acid sequences of phage-displayed peptides selected in step (b).

14. The method of claim 11, wherein said chemically-synthesized peptide is conjugated to a carrier.

15. The method of claim 14, wherein said carrier is a protein.

16. The method of claim 14, wherein said carrier is a synthetic polymer.

17. The method of claim 16, wherein said polymer consists essentially of branched polylysine.

18. The method of any one of claims 1-4, wherein said antigen is L-selectin.

19. The method of claim 18, wherein said L-selectin is human L-selectin.

20. The method of claim 19, wherein said mammal is a human antibody-transgenic mouse.

21. The method of claim 4, wherein said antigen is human L-selectin and said phAbs function to inhibit lymphocyte binding to endothelial venules.

22. A method of making a human antibody that is specific for a desired epitope of a chosen antigen, comprising the steps of:
(a) biasing the immune response of a human antibody-transgenic mammal toward said epitope according to the method of any one of claims 1-4; and then
(b) isolating an antibody from said mammal that is specific for said epitope of said antigen.

23. The method of claim 22, wherein said human antibody-transgenic mammal is a human antibody-transgenic mouse.

24. A human antibody that is specific for a desired epitope of a chosen antigen, produced by the process of claim 23.

25. The antibody of claim 24, wherein said antibody is monoclonal.

26. The antibody of claim 24, wherein said antibody is specific for an epitope of human L-selectin.

27. The antibody of claim 26, wherein said antibody is IgG.

28. The antibody of claim 27, wherein said antibody has an affinity of less than 10⁻⁹ M.

29. The antibody of claim 26, wherein said antibody inhibits binding of lymphocytes to endothelial venules.

30. The antibody of claim 24, wherein said antibody is specific for an epitope of a melanoma-associated antigen.

31. The antibody of claim 30, wherein said antibody is specific for an epitope of the melanoma-associated gp100 antigen.

32. A library of antibodies or antigen-binding antibody fragments, wherein said antibodies or antibody fragments derive from a mammal with immune response biased according to the method of any one of claims 1-4.

33. The library of claim 32, wherein said antibodies are human antibodies.

34. The library of claim 33, wherein said antibody fragments are phage-displayed scFv fragments.

35. The library of claim 33, wherein said antibody fragments are phage-displayed Fab fragments.

36. The library of claim 33, wherein said antibody fragments are soluble scFv fragments.

37. The library of claim 33, wherein said antibody fragments are soluble Fab fragments.

38. The library of claim 33, wherein said antibodies are heterodimeric IgG/K antibodies.

39. A method for generating an epitope-expression profile of a given protein, comprising:
(a) contacting a plurality of biased antibody libraries with said protein;
(b) detecting the binding of said protein to the antibodies of said libraries;
(c) collecting said binding data into a single data structure.

40. A method for generating a human-like antibody having a desired function against a target molecule, comprising:
(a) providing a panel of human antibody moieties that are derived from human antibody transgenic non-human animals that are immunized with cells representing selected tissues;
(b) probing the panel of antibody moieties with the target molecule and selecting antibody moieties that bind to the target molecule with an affinity greater than 10⁻⁸ M;
(c) functionally assessing the selected antibody moieties from the probing step for the desired function and selecting those antibody moieties that possess the desired function;
(d) screening the antibody moieties selected in the functionally assessing step with peptides to determine and select mimotopes of the target molecule;
(e) immunizing a human antibody transgenic non-human mammal with mimotopes selected in the screening step; and
(f) recovering human-like antibody moieties from the transgenic mammal that bind to the target molecule with an affinity greater than 10⁻⁸ M and possess the desired function against the target molecule.
